(19) United States
(12) Patent Application Publication
(10) Pub. No.: US 2003/0171657 A1
(43) Pub. Date: Sep. 11, 2003

(54) Selection of optimal medication methodology (SOOMM)

(76) Inventors: Ralph Leonard, New Haven, CT (US); Robert Robinson, Springfield, IL (US)

Correspondence Address: Ralph Leonard, Apt 400, 1145 Chapel Street, New Haven, CT, 06510, US

(21) Appl. No.: 10/054,504

(22) Filed: Jan. 22, 2002

(51) Int. Cl. A61B 5/00; G06F 17/60

(52) U.S. Cl. 600/300; 705/2

(57) ABSTRACT

The process by which a clinician selects what s/he thinks is the best medication for a patient with a given disease is at best an estimation of the myriad of risks and benefits each may have, and at worst a subjective guess. Because of burgeoning classes of medications and their members, the associated risks and benefits of each class and medication, and the addition of external medical guidelines, this task is becoming very difficult and at times dangerous. We describe a process that allows one to approach this problem in a systematic, quantitatively objective manner and include any number of facts and decision rules without relying solely on complex algorithms. The process easily accommodates fact revisions without redesigning algorithms, thus updates quickly. Although intuitive, the process would be very tedious to do manually but lends itself well to automated computations and medical software.

### CROSS-REFERENCE TO RELATED APPLICATIONS

To the best of our knowledge, there are no prior patents which describe the process which we are submitting for application. However, a number of patents do mention in some context the problem which we are addressing, in varying levels of detail. Those patents are listed in the following table.

| Patent | Date of | First | |
|---|---|---|---|
| number | patent | Author | Title |
| H958 | Aug. 6, 1991 | DeVita, Jr | PDQ cancer treatment |
| | | | information system |
| 3,946,503 | Mar. 30, 1976 | Buchan; | Audio-visual apparatus and |
| | | Robert R. C. | method of using the apparatus |
| | | | for obtaining computer |
| | | | compatible data from an |
| | | | individual |
| 4,370,983 | Feb. 1, 1983 | Lichtenstein; | Computer-control medical |
| | | Eric S. | care system |
| 4,839,822 | Jun. 13, 1989 | Dormond, | Computer system and method |
| | | Kenneth | for suggesting treatments for |
| | | | physical trauma |
| 4,895,518 | Jan. 23, 1990 | Amold | Computerized diagnostic |
| | | | reasoning evaluation system |
| 4,899,758 | Feb. 13, 1990 | Finkelstein | Method and apparatus for |
| | | | monitoring and diagnosing |
| | | | hypertension and congestive |
| | | | heart failure |
| 5,054,493 | Oct. 8, 1991 | Cohn, Jay N. | Method for diagnosing, |
| | | | monitoring and treating |
| | | | hypertension |
| 5,072,383 | Dec. 19, 1991 | Brimm, | Medical information system |
| | | John E. | with automatic updating of |
| | | | task list in response to |
| | | | entering orders and charting |
| | | | interventions on associated |
| | | | forms |
| 5,077,666 | Dec. 31, 1991 | Brimm, | Medical information system |
| | | John E. | with automatic updating of |
| | | | task list in response to |
| | | | charting interventions on task |
| | | | list window into an associated |
| | | | form |
| 5,835,900 | Nov. 10, 1998 | Fagg, III; | Computer-implemented |
| | | Fred D | decision management system |
| | | | with dynamically generated |
| | | | question sand answer choices |
| 5,935,060 | Aug. 10, 1999 | Iliuff; | Computerized medical |
| | | Edwin C | diagnostic and treatment |
| | | | advice system including list |
| | | | based processing |
| 5,960,419 | Sep. 28, 1999 | Fagg, III; | Authoring tool for computer |
| | | Fred D | implemented decision |
| | | | management system |
| 5,961,332 | Oct. 5, 1999 | Joao; | Apparatus for processing |
| | | Raymond | psychological data and |
| | | | method of use thereof |
| | | Anthony | |
| 5,963,931 | Oct. 5, 1999 | Fagg, | Computer-assisted decision |
| | | Fred D. | management system |
| 5,987,784 | Nov. 2, 1999 | Fagg, | Computer-implemented |
| | | Fred D. | decision management system |
| | | | with dynamically generated |
| | | | questions and answer choices |
| 5,993,386 | Nov. 30, 1999 | Ericsson; | Computer assisted method for |
| | | Arthur Dale | the diagnosis and treatment of |
| | | | illness |
| 6,009,420 | Dec. 28, 1999 | Fagg, | Computer-implemented |
| | | Fred D. | decision management system |
| | | | with dynamically generated |
| | | | questions and answer choices |
| 6,047,259 | Apr. 4, 2000 | Campbell, | Interactive method and system |
| | | Scott | for managing physical exams, |
| | | Douglas | diagnosis and treatment |
| | | | protocols in a health care |
| | | | practice |
| 6,063,028 | Mar. 20, 1998 | Luciano, | Automated treatment selection |
| | | Joanne | method |
| | | Sylvia | |
| 6,081,786 | Jun. 27, 2000 | Barry, | Systems, methods and |
| | | David W. | computer program products |
| | | | for guiding the selection of |
| | | | therapeutic treatment |
| | | | regimens |
| 6,112,750 | Sep. 5, 2000 | Chandra | Method and system for |
| | | | assessing risks and prognoses |
| | | | of a given course of medical |
| | | | treatment |
| 6,188,988 | Feb. 13, 2001 | Barry, | Systems, methods and |
| | | David W. | computer program products |
| | | | for guiding the selection of |
| | | | therapeutic treatment |
| | | | regimens |
| 6,208,974 | Mar. 27, 2001 | Campbell | Method and system for |
| | | | managing wellness plans for a |
| | | | medical care practice |
| 6,260,035 | Jul. 10, 2001 | Horvitz, | intelligent user assistance |
| | | Eric | facility for a software program |
| 6,269,339 | Dec. 29, 1998 | Silver, | System and method for |
| | | Charles | developing and selecting a |
| | | | customized wellness plan |

### STATEMENT REGARDING FEDERALLY SPONSORED RESEARCH OR DEVELOPMENT

There were no direct or indirect federally sponsored research money or support given for the development of this application or idea.

### REFERENCE TO A MICROFICHE APPENDIX

Does not apply

### BACKGROUND OF THE INVENTION

Definitions

For the purposes of the rest of this application, we will use the following terms as defined here. 1) Therapeutic Class: A group of therapies (which may be pharmaceutical or non-pharmaceutical therapies) with similar therapeutic benefits and hazards (called indications and contraindications, respectively) such that they are considered homogenous and can be used interchangeably under many circumstances. A therapeutic class can contain one or more medications. While the Food and Drug Administration (FDA) is the predominant government body that defines members of a medication class in the US, for the purposes of this program any source may be chosen by the program implementer to define members of a class. 2) Medical Condition—The presence of a diagnosed disease state, laboratory test abnormality, or physical exam abnormality; the latter two may not necessarily be diagnosis but rather physical finding abnormalities.

When choosing a therapy for a given patient (for simplicity, from here onwards we will assume the therapy is a medication), the physician must consider the various risks and benefits of the available medication classes and then the specific medications within the class. Risks and benefits are more formally called “contraindications” and “indications”, respectively. This process, though seemingly intuitive, can become remarkably complex because each contraindication and indication may be graded as relative (low) or absolute (high) thus there can be different weights in comparing each choice.

Furthermore, the number of choices of both medication classes and the medications within those classes are growing at a rate that makes such a consideration of all the choices nearly impossible for a human or automated machine (such as a computer) to do without a formal approach.

National authorities have published guidelines which attempt to simplify this process and employ an algorithm approach. The approach, while lending itself easily to comprehension by humans and software design by programmers, captures only the major decision elements in choosing a medication and may be of little or no help in deciding among many seemingly equal choices.

Unfortunately, because patients so often have many co-morbid medical conditions, there is a staggering number of combinations of medication indications and contraindications for each patient such that any system (such as a nueral network or inference engine) that attempts to include all relevant possibilities is overwhelmed. For example, if there were only 100 relevant diseases and a given patient could only have 4 diseases, there would be 3.9 million possible disease combinations (irrespective of sequence); this is a large list of specific entities to account for yet without analyzing each one it is difficult to be sure the best medications would be chosen. In reality there are thousands of diseases and patients commonly have over a dozen, thus the possibilities become astronomical and can not be preconceived and ranked. Even if there were a way to identify and attribute best scores for each combination, the time needed to maintain the database would be prohibitive and even fast computers would take unacceptably long delays to identify the correct answer.

Furthermore, hierarchical systems which assign greater weights to some decisions (usually earliest ones) oversimplify and distort the problem such that incorrect weight is given to some aspects and the final preference ranking is not correct.

### BRIEF SUMMARY OF THE INVENTION

This invention is a process that can be used manually by humans or automated with computer software to systematically deduce the optimal medications for a given patient based on all known indications and contraindications. In addition, it can incorporate preferences among medication classes as determined by the guideline publishing authority for a given disease. Finally, because it does not involve complex decision trees, modifications can be incorporated in the process without extensive revisions of the algorithm which can greatly simplify and expedite programming updates

The process addresses the many indications and contraindications systematically by using a matrix for each medication class, and depending upon the patients co-morbidities, s/he will be best described by one or more cells in the matrix, then assigned the numeric value of that cell (in the case of fulfilling more than one cell, the value assigned for that medication class is the lowest cell value). As described in greater detail below, this is simplified if one assumes that a higher cell value represents a high benefit and no risk. Once this process has been repeated for all potentially eligible medication classes, one can then select the medication class to which the patient's represented cell value is greatest, or depending upon external rules from an algorithm, choose from among intermediate valued cell classes which may have essentially equal values.

To do this process manually is not practical, especially in the setting of a busy clinic practice. However, because it is the summation of many true/false computations, it lends itself very naturally to automated computation and is suited to software designed to help clinicians make complex decisions quickly.

### BRIEF DESCRIPTION OF THE SEVERAL VIEWS OF THE DRAWING

Does not apply.

### DETAILED DESCRIPTION OF THE INVENTION

In this section we will illustrate the concept with a theoretical example and will divide the process into two steps.

For the purposes of this discussion, a matrix will be defined as a table with four columns (vertical) orthogonal to four rows (horizontal) that comprise sixteen cells.

Step 1: Find the Best Medication Class.

The first assumption is that all medications may impart both a benefit and a harm (almost always true) and that the extent of the benefit and harm is largely due to the concomitant diseases of the given patient. We use the terms “indications” and “contraindications” to denote the benefit and harm in a patient with a given disease.

The second assumption is that both the indications and contraindications may be categorized into three levels: none, moderate and high. One can then get a composite score of how the multiple pro and contra considerations balance regarding a given medication class for a patient with a set of various diseases. This is done by using a simple 4×4 cell matrix where indications and contraindications are along the axes, as in Figure A.

[image]

If one assumes that a mild contraindication and a mild indication are equal, then the 9 ordinal values assigned to the matrix may fall as follows in Figure B.

[image]

However, most practicing physicians would not equate a high indication with a high contraindication; in fact, most would consider the “risks” of a high contraindication to far outweigh the “benefits” of a high indication, thus abiding by the dictum “First, do no harm.” Therefore, we think a matrix as in Figure C with the following values is a more representative approach.

[image]

One can quantify the degree of indication and contraindication by using score for each, then plot those scores on a table such as in Figure D.

[image]

For example, if an individual has one disease which is an absolute contraindication for a medication then their Vertical Variable Score (WS) is 1; likewise, if s/he has another indication which is a relative (moderate) indication then the Horizontal Variable Score (HVS) is 2. The Final Variable Score (FVS) is the cell which has the intersection of the HVS and the VVS; in this example the FVS would be 2. This is analogous to defining a point in the right upper quadrant, in relation to the origin (0,0), of a Cartesian plane using positive coordinates on the ordinate and abscissa axis, respectively.

The above example illustrates how a specific cell may be located and be denoted the composite score of the indications and contraindications for that medication class. This composite medication class score is called the Final Variable Score (FVS) and may be reached for a given medication class applied to an individual patient. This process can be repeated for each possible class of medications used to treat a given disease thus arriving at several FVSs.

To pick between classes may then be based on consideration of their FVSs but also allows these classes to be presented in a preferred selection method so that the first which is equal to or greater than a given value will be chosen. This is illustrated in Figure E where we see that, although medication X has a lower score than Y, it is still preferred because of additional selection criteria that are easily applied. This is frequently the case in medicine where guidelines suggest that, all other things being equal, one medication class should be used before another (e.g., in treating hypertension one should use a diuretic before an ACE-Inhibitor).

[image]

Figure E shows that the highest score “wins”, but ties may be broken by external rules such as from guidelines (thus Class W was placed ahead of Class X). However, one could also specify that if a minimum score is met (e.g., 6) then an external order is forced among those with equal or greater than those scores irrespective of the absolute values.

One can also identify a quantitative threshold below which no choice is considered safe. For example, one might say that values of 5 or less, as displayed in Figure C, require that no recommendation is offered automatically and the patient and physician must discuss the risks and benefits in greater detail.

While this process described above may seem intuitive and can be easily programmed in any computer language, the actual number of considerations is rather daunting. For example, each class of medication involves the determination of up to 9 calculations so one can assign the appropriate FVC, (e.g., each calculation could use an If-then clause to search for diseases in a patient's problem list and yield a number if it is met) thus if faced with the question of routine hypertension for where seven classes are available (ACE-I, BB, CCB, A2B, diuretics, centrally acting medication, vasodilators) , this involves 63 calculations plus the process of sorting between classes for a total of 64 computations.

For patients who have multiple indications and contraindications, their FVC could be more than one value for a given medication class. To avoid ambiguity and error on the conservative side, we use a descending order search such that the lower of more than one values will be assigned to that medication class.

Part 2: Picking a Medication within a Class.

Once the ideal medication class has been identified, the process of picking a given member is relatively easy since such decisions are primarily based upon arbitrary factors (mostly financial, due to negotiations between an insurance agency and a pharmaceutical company) since a class for our purposes requires internal homogeneity. The specific medication is usually selected from in ascending order of the provider's list (e.g., choose the most preferred first).

### Sample Code

The sample code here is only relevant to this process patent application. There are many other considerations in medication management (e.g., suggesting discontinuing of less favorable medication, checking f6r medication interactions, recommending frequency or dose adjustments of selected medications) all of which are relatively simple and beyond the scope of this process patent application.

Rather than write this code in one programming language, we have done it using “pseudocode” in the structured programming format. This allows the concepts to be more transparent and readily translated to any specific language. We are using the following assumptions of standard pseudocode language

1) A single apostrophe precedes code that explains the function of a group of code.

2) The computer processes the program from top to bottom (The place of this code is dependent upon the programming language used (e.g., for Visual Basic and many programs based on Windows, it will be triggered by an event) but that is irrelevant to the actual code content)

3) Indentations imply grouped operations which may be performed as loops or code to be executed only when the immediately preceding condition is true

4) “If, Then” operations are expressed as ‘If, then’ if true and an indented ‘otherwise’ if false.

We have inserted text in arial font such as this, to explain some of the rationale that would not normally be included in the code, even preceded by a single apostrophe. This is to aid comprehension.

For the purposes of illustration, we are using the following hypothetical medication classes and diseases as written in Tables 1 and 2. This is a gross oversimplification since there are thousands of medications and diseases. We have given these symbolic representation but also, for those familiar with some medicine, actual class names in parenthesis. The names are only to make the methods more apparent and, since the medical knowledge and truth of these tables may change, are not absolute but merely illustrative.

TABLE 1

| Medication class* | Specific medications in class* |
|---|---|
| A (ACE-I) | A1, A2, A3, A4, A5 |
| B (BB) | B1, B2, B3, B4, B5 |
| C (CCB) | C1, C2, C3, C4, C5 |
| D (Diuretic) | D1, D2, D3, D4, D5 |
| E (SSRI) | E1, E2, E3, E4, E5 |
| F (B-agonist) | F1, F2, F3, F4, F5 |
| G (warfarin) | G1, G2, G3, G4, G5 |

*Medication classes are designated with letters and medications within a class are designated as numbered letters. The words in parenthesis are medication class names or their acronyms.

TABLE 2
Key Of Disease And Class Name Abbreviations

| Number | Class or Disease fully delineated |
|---|---|
| A (ACE-I) | Angiotension Converting Enzyme Inhibitor |
| B (BB) | Beta-blocker |
| C (CCB) | Calcium channel blocker |
| D (Diuretic) | Thiazide diuretic |
| E (SSRI) | Selective serotonin re-uptake inhibitor |
| F (B-agonist) | Beta-agnost |
| G (warfarin) | Generic name of a specific anti-coagulant. |
| 1 | Essential systemic hypertension |
| 2 | Left ventricular systolic dysfunction (NYH class III, in this |
| | example) |
| 3 | Proteinuria |
| 4 | Within 6 months of myocardial infarction |
| 5 | Supraventricular tachycardia 2^{nd} to atrial fibrillation |
| 6 | Migraine headaches |
| 7 | Non-psychotic unipolar major depression (moderate |
| | severity) |
| 8 | Non-psychotic unipolar major depression (high severity) |
| 9 | Obsessive-compulsive disorder |
| 10 | Generalized panic disorder |
| 11 | Reversible reactive airway disease (moderate severity) |
| 12 | Reversible reactive airway disease (high severity) |
| 13 | Myocardial infarction more than 6 months ago |
| 14 | Ischemic stroke |
| 15 | Atrial fibrillation in patients >65 |
| 16 | Prosthetic aortic valve |
| 17 | Bilateral renal artery stenosis |
| 18 | 2^{nd} degree type I atrio-ventricular block |
| 19 | Hyperuricemia |
| 20 | Carcinoid tumor |
| 21 | Allergy to b-agonists |
| 22 | Idiopathic subaortic stensosis (IHSS) |

*Medication Classes are designated with letter, diseases are designated with numbers and medications within a class are designated as numbered letters.

Table 3 contains some examples of indications and contraindications for medications and classes that are essential in generating appropriate scores. For medications which are grouped by the FDA in a given class but are dissimilar in indications or contraindications from other members of a class, a separate class will be generated by the programmers so its properties may be considered in detail (such as with warfarin where it is considered the sole member of its class).

TABLE 3

| Medication | Strong | Moderate | Strong | Moderate |
|---|---|---|---|---|
| class | indication | indication | contraindi- | contraindi- |
| | | | cation | cation |
| A (ACE-I) | 2, 3, 4 | 1 | 17, 22 | |
| B (BB) | 1 | 6, 2, 5, 18 | 7, 11 | 8, 12, |
| C (CCB) | 5 | 1, 18 | | 2 |
| D (Diuretic) | 1 | | | 19 |
| E (SSRI) | 7, 8 | | 20 | |
| F (B-agonist) | 11 | | | 5, 21 |
| G (warfarin) | 15, 16 | | | |

For illustration, let us assume that our sample patient has diseases 1 and 22 and that this segment of the code is just concerned with treating the hypertension (disease 1)

‘ Step 1: find the final variable scores for all medication classes.
‘ Determine the score for every medication class

‘Determine horizontal variable score (HVS) for A, called HVS(A)
If problem list contains diagnosis of “2”, “3” or “4” then HVS(A)=3

Otherwise,
If problem list contains diagnosis of “1” then HVS(A)=2

Otherwise,
HVS(A)=1

‘Determine vertical variable score (VVS) for A, called VVS(A)
If problem list contains diagnosis of “17” or “22” then VVS(A)=1

Otherwise,
VVS(A)=3

In the case when there is an empty set, meaning there are no diagnosis in the a respective cell
(as in the case for moderate contraindications in medication class A), the If-Then and Otherwise
line is dropped for that class.

‘Determine final variable score (FVS) for A, called FVS(A)
If HVS(A)=3 and VVS(A)=3 then FVS(A)=9

Otherwise,
If HVS(A)=2 and VVS(A)=3 then FVS(A)=8

Otherwise,
If HVS(A)=1 and VVS(A)=3 then FVS(A)=7

Otherwise,

If HVS(A)=3 and VVS(A)=2 then (FVS)A=6

Otherwise,
If HVS(A)=2 and VVS(A)=2 then FVS(A)=5

Otherwise,
If HVS(A)=1 and VVS(A)=2 then FVS(A)=4

Otherwise,
If HVS(A)=3 and VVS(A)=1 then FVS(A)=3

Otherwise,
If HVS(A)=2 and VVS(A)=1 then FVS(A)=2

Otherwise,
FVS(A)=1

‘Determine horizontal variable score (HVS) for B, called HVS(B)
If problem list contains diagnosis of “1” then HVS(B)=3

Otherwise,
If problem list contains diagnosis of “2”, “5”, “6” or “18” then HVS(B)=2

Otherwise,
HVS(B)=1

‘Determine vertical variable score (VVS) for B, called VVS(B)
If problem list contains diagnosis of “7” or “11” then VVS(B)=1

Otherwise,
If problem list contains diagnosis of “8” or “12” then VVS(B)=2

Otherwise,
VVS(B)=3

‘Determine final variable score (FVS) for B, called FVS(B)
If HVS(B)=3 and VVS(B)=3 then FVS(B)=9

Otherwise,
If HVS(B)=2 and VVS(B)=3 then FVS(B)=8

Otherwise,
If HVS(B)=1 and VVS(B)=3 then FVS(B)=7

Otherwise,
If HVS(B)=3 and VVS(B)=2 then FVS(B)=6

Otherwise,
If HVS(B)=3 and VVS(B)=2 then FVS(B)=6
Otherwise,
If HVS(B)=2 and VVS(B)=2 then FVS(B)=5

Otherwise,
If HVS(B)=1 and VVS(B)=2 then FVS(B)=4

Otherwise,
If HVS(B)=3 and VVS(B)=1 then FVS(B)=3

Otherwise,
If HVS(B)=2 and VVS(B)=1 then FVS(B)=2

Otherwise,
FVS(B)=1

‘Determine horizontal varibale score (HVS) for C, called HVS(C)
If problem list contains diagnosis of “5” then HVS(C)=3

Otherwise,
If problem list contains diagnosis of “1” or “18” then HVS(C)=2

Otherwise,
HVS(C)=1

‘Determine vertical variable score (VVS) for C, called VVS(C)
If problem list contains diagnosis of “2” then VVS(C)=2

Otherwise,
VVS(C)=3

‘Determine final variable score (FVS) for C, called FVS(C)
If HVS(C)=3 and VVS(C)=3 then FVS(C)=9

Otherwise,
If HVS(C)=2 and VVS(C)=3 then FVS(C)=8

Otherwise,
If HVS(C)=1 and VVS(C)=3 then FVS(C)=7

Otherwise,
If HVS(C)=3 and VVS(C)=2 then FVS(C)=6

Otherwise,
If HVS(C)=2 and VVS(C)=2 then FVS(C)=5

Otherwise,
If HVS(C)=1 and VVS(C)=2 then FVS(C)=4

Otherwise,
If HVS(C)=3 and VVS(C)=1 then FVS(C)=3

Otherwise,
If HVS(C)=2 and VVS(C)=1 then FVS(C)=2

Otherwise,
FVS(C)=1

‘Determine horizontal variable score (HVS) for D, called HVS(D)
If problem list contains diagnosis of “1” then HVS(D)=3

Otherwise,
HVS(D)=1

‘Determine vertical variable score (VVS) for D, called VVS(D)
If problem list contains diagnosis of “19” then VVS(D)=2

Otherwise,
VVS(D)=3

‘Determine final variable score (FVS) for D, called FVS(D)
If HVS(D)=3 and VVS(D)=3 then FVS(D)=9

Otherwise,
If HVS(D)=2 and VVS(D)=3 then FVS(D)=8

Otherwise,
If HVS(D)=1 and VVS(D)=3 then FVS(D)=7

Otherwise,
If HVS(D)=3 and VVS(D)=2 then FVS(D)=6

Otherwise,
If HVS(D)=2 and VVS(D)=2 then FVS(D)=5

Otherwise,
If HVS(D)=1 and VVS(D)=2 then FVS(D)=4

Otherwise,
If HVS(D)=3 and VVS(D)=1 then FVS(D)=3

Otherwise,
If HVS(D)=2 and VVS(D)=1 then FVS(D)=2

Otherwise,
FVS(D)=1

‘Determine horizontal variable score (HVS) for E, called HVS(E)

If problem list contains diagnosis of “7” or “8” then HVS(E)=3

Otherwise,
HVS(E)=1

‘Determine vertical variable score (VVS) for E, called VVS(E)
If problem list contains diagnosis of “20” then VVS(E)=1

Otherwise,
VVS(E)=3

‘Determine final variable score (FVS) for E, called FVS(E)
If HVS(E)=3 and VVS(E)=3 then FVS(E)=9

Otherwise,
If HVS(E)=2 and VVS(E)=3 then FVS(E)=8

Otherwise,
If HVS(E)=1 and VVS(E)=3 then FVS(E)=7

Otherwise,
If HVS(E)=3 and VVS(E)=2 then FVS(E)=6

Otherwise,
If HVS(E)=2 and VVS(E)=2 then FVS(E)=5

Otherwise,
If HVS(E)=1 and VVS(E)=2 then FVS(E)=4

Otherwise,
If HVS(E)=3 and VVS(E)=1 then FVS(E)=3

Otherwise,
If HVS(E)=2 and VVS(E)=1 then FVS(E)=2

Otherwise,
FVS(E)=1

‘Determine horizontal variable score (HVS) for F, called HVS(F)

If problem list contains diagnosis of “11” then HVS(F)=3

Otherwise,
HVS(F)=1

‘Determine vertical variable score (VVS) for F, called VVS(F)
If problem list contains diagnosis of “5” or “21” then VVS(F)=2

Otherwise,
VVS(F)=3

‘Determine final variable score (FVS) for F, called FVS(F)
If HVS(F)=3 and VVS(F)=3 then FVS(F)=9

Otherwise,
If HVS(F)=2 and VVS(F)=3 then FVS(F)=8

Otherwise,
If HVS(F)=1 and VVS(F)=3 then FVS(F)=7

Otherwise,
If HVS(F)=3 and VVS(F)=2 then FVS(F)=6

Otherwise,
If HVS(F)=2 and VVS(F)=2 then FVS(F)=5

Otherwise,
If HVS(F)=1 and VVS(F)=2 then FVS(F)=4

Otherwise,
If HVS(F)=3 and VVS(F)=1 then FVS(F)=3

Otherwise,
If HVS(F)=2 and VVS(F)=1 then FVS(F)=2

Otherwise,
FVS(F)=1

‘Determine horizontal variable score (HVS) for G, called HVS(G)

If problem list contains diagnosis of “15” or “16” then HVS(G)=3

Otherwise,
HVS(G)=1

‘Determine vertical variable score (VVS) for G, called VVS(G)
VVS(G)=3 ‘since there are empty sets in the other categories.
‘Determine final variable score (FVS) for G, called FVS(G)
If HVS(G)=3 and VVS(G)=3 then FVS(G)=9

Otherwise,
If HVS(G)=2 and VVS(G)=3 then FVS(G)=8

Otherwise,
If HVS(G)=1 and VVS(G)=3 then FVS(G)=7

Otherwise,
If HVS(G)=3 and VVS(G)=2 then FVS(G)=6

Otherwise,
If HVS(G)=2 and VVS(G)=2 then FVS(G)=5

Otherwise,
Of HVS(G)=1 and VVS(G)=2 then FVS(G)=4

Otherwise,
If HVS(G)=3 and VVS(G)=1 then FVS(G)=3

Otherwise,
If HVS(G)=2 and VVS(G)=1 then FVS(G)=2

Otherwise,
FVS(G)=1

For this particular patient, the scores are FVS(A)=2, FVS(B)=7, FVS(C)=7, FVS(D)=9,
FVS(E)=7,FVS(F)=7, FVS(G)=7.

‘ Step 2: Now restrict the focus to just those medication classes that can be used
to treat the disease of intrest, in this case disease 1.

If problem list does not include, “1” then skip this section.

Otherwise,

‘Sort medication classes by FVS in descending order. In the case in which the
final variable scores for more than one medication class to equal, then sort
the medication class scores according to assigned guideline preferences. In
this case we are told that the preferred order for treating this disease is
‘d’>’b’>’a’=’c’ (i.e., ‘d’ is preferred to ‘b’, ‘b’ is preferred to ‘a’ and ‘a’
and ‘c’ are equally preferred).

Do primary sort the FVS for all 8 classes in a single, descending list using

loops.

In the case of ties, do a secondary sort using loops such that ‘d’>’b’>’a’=’c’
Let the preferred medication class (PMC) = the FVS that is first in the sorted

order of the final (primary enhanced with secondary sort) list.

If the FVS of the PMC is <=5 then display text “there is no medication class

which offers benefits which outweigh its risk. The computer will not suggest
any class and deferes to the preferences of the clinician and patient.’

Otherwise,
Display the text “The preferred medication class is “PMC”.”

‘Now choose a specific medication (SM) within the preferred class.

Let SM= the medication name within the PMC that is first on the list germane

to that patient's other variable constraints. In particular, each 3rd party
payer will arrange specific medications within a class based on cost
preferences and that order should be honored.

Display the text “The preferred medication for this patient is “SM”.”
Display as choices in a list (e.g., a drop-down list or list-box) the

starting, usual therapeutic and maximum doses recommended by the FDA for the
SM when treating Hypertension.
For our sample patient who does not have disease ‘1’ (hypertension), the PMC is ‘D’ (thiazide
diuretic), and depending upon her/his insurance, a specific name and dose could be listed, such
as “hydrochlorathiazide 12.5 mg/day.”
Subsequent code may be written for other diseases for which there is a desire to find the optimal
medication and thus repeat step 2 many times.

For our sample patient who does have disease ‘1’ (hypertension), the PMC is ‘D’ (thiazide diuretic), and depending upon her/his insurance, a specific name and dose could be listed, such as “hydrochlorathiazide 12.5 mg/day.”

Subsequent code may be written for other diseases for which there is a desire to find the optimal medication and thus repeat step 2 many times.

The above code is simply one example of how this process may be expressed in a computer language. It is not to be construed as the only means and is purely illustrative. Accordingly, modifications by those skilled in the trade of computer software development, programming languages and logic symbols are possible but may be included within the scope of this invention as defined in the claims.

I. We claim as our invention the systematic process of assigning a quantitative score to each combination of medical conditions the risks and benefits (indications and contraindications, respectively) of all relevant pharmaceutical and non-pharmaceutical classes of therapies for a given disease process described above as Selection Of Optimal Medication Methodology.
A. Sub-claim: the SOOMM system as described in claim I may use from four to an infinite number of cells within a matrix to determine various nuances of indications and contraindications, but the suggested system is for 4×4 with a 3×3 inner space of 9 active cell scores.
B sub-claim: the soomm system as described in claim 1 uses the term class to refer to a group of members within a therapeutic category which are considered homogenous enough to share the same indication and contraindication scores, and may be categorized based on existing classifications (e.g., as proposed by the fda) but also based on de novo schemes created by those who implement it with specific details:
C. Sub-claim: the SOOMM system as described in claim I uses the term “medical condition” to refer to any diagnosed disease or highly probably disease as based on objective data extracted from laboratory or other sources.

II. We claim as our inventions that the SOOMM system may be further augmented by additional external rules embodied in authoritative guidelines that ranks classes for their role in a given disease, all other things being equal.

III. We claim as our invention that the SOOMM process of choosing the best therapeutic class may be applied to therapies within a class where specific distinguishing indications and contraindications exist. The lowest acceptable limit of risks and benefits may be determined by the end-user, but in the 9-cell model illustrated here we recommend values of 5 or lower be cause for giving no recommendation and allowing the clinician to chose a therapy.

IV. We claim that the SOOMM system only requires the patient's medical conditions be entered in such a way that the system may be applied (e.g., populate specific data fields or other).

V. We claim as our invention that the subsequent choices between products within a given class chosen by the SOOMM system (i.e., those in a class are considered equal), may be further ranked according to other factors such as a third-party payer's preferences, cost of the medication and the user's prior picks. Such subsequent ranking may be done after a class is selected by further input by the user (who selects how things are sorted).

VI. We claim as our invention that the SOOMM system may be implemented in a variety of computational devices, as either software or hardware. This implementation can be executed on mainframe, personal (desktop or laptop) computers, mobile computing devices (such as Personal Digital Assistants or mobile phones), delivered via wired or wireless network technology using servers or their equivalents via an intranet or internet.

VII. We claim as our invention that the actual user interface could be graphical or non-graphical, that multiple pointing (mouse, stylus, light-pen etc.) and data entry modalities (keyboard, speech to text conversion, electronic importing, etc.) can also be supported.

VIII. We claim as our invention that the SOOMM system could be written within a broader program such as an electronic medical record designed by another group.

IX. We claim as our invention that the SOOMM system could be written as a stand-alone program that could ask the user or others with information and who are permitted to enter it and that other relevant data may be imported electronically, scanned or via other means such that a complete list of the patient's medical conditions and problems is maintained.

X. We claim as our invention that the SOOMM system can be applied to patients with multiple medical conditions, diagnoses and concomitant medications.

XI. We claim as our invention that the SOOMM system can use, for medical conditions, laboratory or other objective data that defines a medical condition rather than having that stated only in a problem list.

XII. We claim as our invention that the SOOMM system is fundamentally different than an inference engine, neural network or those less rigorous and predictable such as “fuzzy logic” that employ random or statistical probabilities rather than rigorous protocols.

XIII. We claim as our invention that the SOOMM system can be written in any computer programming language, either existing or future developed, that can express the concepts described.

XIV. We claim as our invention that the SOOMM system could run on any operating system that can in turn support a computer programming language.

XV. We claim as our intervention that the SOOMM system may be updated such that the rules may reflect the accepted knowledge of the day and would not be influenced by the prior choices of the user. However, where there is more than one popular recommendation (as in the case when multiple guidelines exist but are contradictory), the user may be given the option of selecting which guidelines s/he wishes to have used in the SOOMM.

XVI. We claim as our invention that the specific recommendations given by SOOMM may only be as accurate as the data on which they are based. Thus, if the accuracy of published research is poor and has been wrongly adopted into recommendations, FDA statements and other data, this will be reflected in the SOOMM recommendations. However, such a limitation is inherent in any medical field and would not be accentuated by SOOMM.

XVII. We claim as our invention that the SOOMM system could be applied to classes of pharmaceutical and non-pharmaceutical treatments that exist or are not discovered, invented or patented once indication and contraindication ratings have been assigned.

XVIII. We claim as our invention that the SOOMM system could be applied to any disease and medical conditions that exist or may be discovered in the future.

XIX. We claim as our invention that the dose of a therapy/medication within a class selected by the SOOMM system may be further modified or suggested based on other parameters provided by manufacturers.

XX. We claim as our invention that the SOOMM system may have additional rules placed upon the selection of therapies from the first ranking of therapies such that additional therapies may be added (e.g., would take the highest score therapy if there were no therapies of any class for given problem; would take the second highest score if a patient were already on the best therapy at a maximal or adequate dose and target control is still not being reached) or lesser ranking ones identified and suggested to be deleted.
